(19) United States
(12) Patent Application Publication
(10) Pub. No.: US 2010/0324428 A1
(43) Pub. Date: Dec. 23, 2010

(54) METHOD AND DEVICE FOR THE NON-INVASIVE MEASUREMENT OF DYNAMIC CARDIOPULMONARY INTERACTION PARAMETERS

(75) Inventor: Ulrich Pfeiffer, Munich (DE)

Correspondence Address: Leydig, Voit & Mayer, Ltd. (Frankfurt office), Two Prudential Plaza, Suite 4900, 180 North Stetson Avenue, Chicago, IL, 60601-6731, US

(73) Assignee: UP-MED GMBH, Munich (DE)

(21) Appl. No.: 12/866,600

(22) PCT Filed: Feb. 13, 2009
(86) PCT No.: PCT/EP09/01031

§ 371 Date: Aug. 6, 2010

(30) Foreign Application Priority Data
Feb. 13, 2008 (DE) 10 2008 008 840.4

(51) Int. Cl. A61B 5/022

(52) U.S. Cl. 600/490

(57) ABSTRACT

A method for a non-invasive determination of cardiopulmonary interaction parameters in a patient includes fitting a pressure cuff on the patient, setting a volume of the pressure cuff in a pulsatile range of the patient, measuring pulsatile signals over time, and evaluating the measured pulsatile signals so as to ascertain the cardiopulmonary interaction parameters.

### CROSS REFERENCE TO PRIOR APPLICATIONS

This application is a U.S. National Phase Application under 35 U.S.C. §371 of International Application No. PCT/EP2009/001031, filed on Feb. 13, 2009, which claims benefit to German Application No. DE 10 2008 008 840.4, filed on Feb. 13, 2008. The International Application was published in German on Aug. 20, 2009 as WO 2009/100927 under PCT Article 21(2).

### FIELD

The invention relates to a method and a device for the non-invasive measurement of dynamic cardiopulmonary interaction parameters.

### BACKGROUND

In clinical medicine, above all in the case of critically ill patients, it is necessary to regularly influence the cardiovascular system in a therapeutically targeted manner. If the cardiovascular system is failing, the decisive question normally arises as to how advisable it is to supplement the circulation by means of infusion solutions or whether alternatively or to what extent the circulation should be supported by circulation-active medicaments. The term “volume responsiveness” or VR is used in this connection.

It has become increasingly clear that the conventional measurement variables for filling the circulatory system, such as e.g. the central venous pressure or also the pulmonary capillary wedge pressure, are not very suitable for predicting volume responsiveness (VR). Although volumetric measurement variables such as e.g. volumes of the heart cavities or the total volume in the ribcage (intrathoracic blood volume) are in principle more suitable, they are also subject to various limitations.

Unlike these more static measurement variables, dynamic measurement variables have therefore recently increasingly been the subject of scientific studies usually based on the interaction of heart and lungs. The pressure fluctuations that are brought about in the ribcage by breathing, in particular during mechanical ventilation with intermittently positive pressures, influence the filling of both the right and the left side of the heart. As a result, a respiration-induced (respiratory) variation of the left-ventricular stroke volume (“stroke volume variation” (SVV)) again results, which is likewise expressed in a respiratory variation of the arterial blood-pressure curve (“pulse-pressure variation” (PPV)), as well as in a variation of the time delay between left-ventricular electrical activity and left-ventricular ejection phase (“pre-ejection phase variation” (PEPV)). It was possible to show that many of the named dynamic cardiopulmonary interaction parameters can predict volume responsiveness better than conventional static cardiovascular measurement variables. However, a disadvantage of almost all VR indices is that they have until now been mostly based on the invasive measurement of the arterial blood pressure and thus require the laborious cannulation of an arterial vessel.

In the article “Relation between respiratory variations in pulse oximetry plethysmographic waveform amplitude and arterial pulse pressure in ventilated patients” by Maxime Cannesson et al. in Critical Care 2005, 9, a non-invasive plethysmographic method was described in which the change in the volumetric flow in the finger was deduced from the pulse oximetry photoplethysmogram. The problem with this method is that it can barely be calibrated and that the intra- and the interindividual reproducibility have not yet achieved the necessary accuracy. In addition, the pulse oximetry method assumes a good blood circulation at the measurement site, which is often not the case precisely in the case of the predominantly customary measurement on the finger in the case of circulatory shock conditions.

### SUMMARY OF THE INVENTION

An aspect of the present invention is therefore the non-invasive detection of the dynamic cardiopulmonary interaction parameters (CIPs).

In an embodiment, a method for the non-invasive determination of in particular dynamic cardiopulmonary interaction parameters (CIPs) in a patient includes the steps: fitting a pressure cuff (20), setting the volume of the pressure cuff in the pulsatile range of the patient, measuring pulsatile signals over time, evaluating the measured pulsatile signals to ascertain the cardiopulmonary interaction parameters (CIPs).

The dynamic cardiopulmonary interaction parameters (CIPs) such as for example PPV, SVV, PEPV as well as further derived variables based on cardiopulmonary interaction can be determined with this method without the need for a laborious cannulation of an arterial vessel. These parameters can thereby be determined non-invasively.

Preferably, this method is used in the case of a ventilated patient, in particular a patient ventilated in a controlled manner. These parameters can provide important information in the case of this patient ventilated in a controlled manner, as here volume shifts are created because of the expended pressure on lungs and indirectly the vessels as well as the heart of the patient.

When fitting a pressure cuff, a pneumatic or hydraulic cuff is preferably used with the aid of which the pulsatile arterial blood pressure fluctuations are detected in a similar manner to the known oscillometric blood pressure measurement on extremities of the body, such as for example arm or leg. Such a pressure cuff can preferably be filled with a fluid.

The pressure of this cuff is then set for this pressure cuff by changing the volume. The volume can be increased by supplying filling material, such as air, fluid, in particular liquid, etc., and the pressure exerted by the cuff on for example the upper arm can thus be increased. The exerted pressure can be reduced by draining off filling material. It is thus preferably also possible to set not the volume but the pressure in the cuff in such a way that an indirect coupling to the volume fluctuations of the arterial blood vessels occurs due to a compression of the respective body part.

Preferably, the volume or the pressure of the pressure cuff is set such that the exerted pressure is selected in such a way that the cuff exerts the pressure between the systolic and the diastolic pressure in the pulsatile range of the patient. In this range, the amplitude of the pulsatile signals is at its highest and thus to be detected most clearly.

The measurement of pulsatile signals then takes place via the pressure fluctuations present in the cuff over time. These can be transmitted to the cuff by the pressure fluctuations caused by the pulse and picked up there. These signals are strongly attenuated compared with the blood pressure signals picked up during an invasive measurement of the arterial blood pressure, as they are measured indirectly via the pressure cuff. These signals are thus preferably picked up indirectly from outside. This preferably happens over time, with the result that a series of measured values is present at specific times.

In a preferred embodiment example, a device with a pneumatic or hydraulic cuff is used with the aid of which the pulsatile arterial blood pressure fluctuations are detected in a similar manner to the known oscillometric blood pressure measurement on extremities of the body. Unlike the oscillometric blood pressure measurement, in which only the systolic, diastolic and the average blood pressure is determined, the respiratory variation range of the named parameters can be determined with the non-invasive measurement of the CIPs according to the invention.

Preferably, values for carrying out a pulse contour method are derived from the pulsatile signals. The absolute blood pressure values are needed to carry out a pulse contour method. In addition, it is possible to improve the signal quality still further compared with the cuffs for the oscillometric blood pressure measurement of the state of the art, with the result that a type of non-invasive continuous blood pressure measurement likewise becomes possible, including all further analysis possibilities such as e.g. pulse contour processes.

For this, in the case of a corresponding evaluation of the pulsatile signals it can be assumed that the pulsatile signals precisely measured in this way directly correspond to the arterial pressure.

It is also preferably possible to multiply the pulsatile signals by a factor or to use a correction function in order to thereby compensate for the occurring attenuation of the arterial pressure signals. Either this factor can be ascertained empirically by statistical inquiry using a larger set of patients or, alternatively, the factor of the attenuation can then be back-calculated from a direct invasive and simultaneous non-invasive measurement of the pulsatile signals and the evaluation of these signals. This factor can then be consulted in the following non-invasive measurements in order to convert the measured pulsatile signals into the actual current arterial values.

The attenuation which occurs between arterial “true pressure signal” and the pressure signal in the cuff is essentially a function of the compressibility of the tissue. This transmission function can be compensated for in simplified terms by a factor. Basically, it is a transmission function which can be represented e.g. by an equivalent circuit of series and parallel connections of resistors and capacitors, in the simplest case of the parallel connection of a resistor and a capacitor. The numerical compensation of this transmission function is a deconvolution. If the fundamental characteristic of the arterial pressure curve (e.g. based on an idealized model curve) and the fundamental characteristic of the transmission function (e.g. resistor and capacitor in parallel connection) are known, the parameters for transmission function for precisely correcting and back-calculating to the “true intravascular pressure signal” can preferably be ascertained as follows: in a first step the systolic and the diastolic or average arterial pressure is ascertained by means of conventional oscillometric pressure measurement. In a second step the average pressure in the cuff is “clamped” at the pressure at which the maximum pulsatile signal quality is to be recorded (normally at the average arterial pressure). Thus, the parameters of the transmission function which lead to the “best fit” with the arterial model curve are then ascertained by iterative adaptation according to the minimum square deviation method, wherein the systolic pressure value and the diastolic pressure value are predetermined by the previously collected measured values. With an adequate signal quality, a preceding determination of these pressure values can also be dispensed with, and these are jointly determined as free parameters in the iteration process.

In this way, it is possible to use the measured pulsatile signals in order to herewith carry out pulse contour methods to estimate the cardiac volume (CV) or pulse contour stroke volume.

Preferably, more signal energy is transmitted from the arm or the extremity to the measuring unit. The signal-to-noise ratio is thus improved. Thus, the larger the contact surface with the arm (the extremity), the larger the transmission surface and thus also the greater the signal energy that is available.

When the measured pulsatile signals are evaluated, the individual measured values are preferably combined into measured values that are to be allocated to a heartbeat. Moreover, an allocation to a respiratory cycle can also take place. Thus, for example after eliminating artefacts, minimum and maximum of the individual blood pressure fluctuations per heartbeat can then be ascertained and the fluctuations within a breathing cycle are ascertained.

In this way it is possible to determine the desired cardiopulmonary interaction parameters (CIPs).

The basis of the oscillometric blood pressure measurement of the state of the art is in principle that in the case of a pressure cuff in contact from the outside the arterial blood vessels display fluctuations in calibre as long as the cuff pressure is smaller than the systolic and greater than the diastolic blood pressure. These fluctuations in calibre of the arterial blood vessels in turn lead to pulsatile pressure fluctuations in the blood pressure cuff. In the case of a cuff pressure that is greater than the systolic blood pressure, the arterial blood vessels are completely compressed during the whole cardiac cycle and thus no fluctuations in calibre of the vessels and no pulsatile pressure fluctuations in the cuff occur. If the cuff pressure fails to reach the diastolic blood pressure, the arterial blood vessels are completely open during the whole cardiac cycle and likewise no pulsatile fluctuations occur. The actual measurement principle of the oscillometric blood pressure measurement is that the pressure in the cuff is increased until pulsatile pressure fluctuations no longer occur. The pressure is then mostly continuously reduced and in the process the pressure values in the cuff at which pulsatility begins, is at its maximum or vanishes are identified. The systolic, diastolic and the average arterial blood pressure are determined from these characteristics.

In the non-invasive measurement of the CIPs it is preferably provided according to the present invention that the systolic and diastolic blood pressure values are determined in advance as marginal values and in addition the variation of these values which are based on the respiratory CIPs.

In a further preferred embodiment example of the present invention, a method is provided in which the cardiopulmonary interaction parameters (CIPs) comprise the stroke volume variation (SVV), the pulse pressure variation (PPV) and/or the pre-ejection phase variation (PEPV). Further derived variables based on cardiopulmonary interaction can also be used as CIPs. Here, the respiratory fluctuation of the pulse wave velocity or the respiratory variation range of the rate of pressure increase are also conceivable.

In a further preferred embodiment example of the present invention, a method is provided in which the pulsatile signals are measured over at least one breathing cycle of the patient, preferably over at least three breathing cycles of the patient. Here, the breathing cycle is preferably determined from the temporal course of the pulsatile fluctuations. Alternatively, the identification of a breathing cycle can, however, also be carried out via other measurement methods, for example from the thoracic electrical impedance signal which can be detected via the ECG electrodes. Preferred further methods for determining the breathing cycle are for example those described in EP 1 813 187. Further advantageous evaluation possibilities for the blood pressure data obtained according to the invention are also given here, to which reference is hereby made. For example, a display of the parameters such as PPV can preferably be suppressed when for example there is an arrhythmia or irregular breathing (no controlled ventilation).

The measurement period preferably comprises at least one respiratory cycle or breathing cycle, preferably several, particularly preferably three or more respiratory cycles. This can be achieved for example by keeping the pressure in the cuff within the pulsatile range over a prolonged period or draining it off very slowly. Preferably, a corresponding control of the volume in the cuff—and thus indirectly of the applied pressure—is provided for this. Unlike the oscillometric blood pressure measurement in which essentially the average pressure in the cuff at the time of the start of pulsatility, at the time of the maximum fluctuations or at the time of the vanishing of pulsatility is decisive, in the CIP method according to the present invention the pulsations themselves are preferably evaluated.

In a further preferred embodiment example of the present invention, a method is provided in which the respiratory variation range of the cardiopulmonary interaction parameters (CIPs) is ascertained.

In a preferred characteristic feature, the maxima and the subsequent minima are determined (amplitude)—alternatively, the minima and the subsequent maxima, i.e. the blood pressure amplitude is ascertained from the systolic and the preceding diastolic pressure—as well as then the amplitude variation over the respiratory cycle as a measure of the pulse pressure variation. In principle, the pulsatile pressure fluctuations in the cuff which are brought about by the pulsatile fluctuations in calibre of the blood vessels are substantially smaller than the pulsatile pressure fluctuations in the arterial blood vessel. However, CIP indices such as the PPV and the SVV are relative measures (they are normally given in %) and the relative percentage variation of the signal relayed into the cuff is closely related to the respiratory variation of the CIP index in the arterial blood vessel. The same is true of the PEPV which is, however, the variation range of a temporal dimension. In this characteristic feature of the CIP measurement method, for example an electrocardiogram for the temporal detection of the start of the electrical cardioactivity can additionally be used for the detection of the time delay between electrical activity and mechanical ejection phase of the heart. The PEPV as CIP index can alternatively also be detected from the time difference between an electrocardiographic and a photoplethysmographic signal.

In a further preferred embodiment example of the present invention, a method is provided in which the volume of the pressure cuff (20) set in the pulsatile range of the patient is kept constant over the measurement of the pulsatile signals. Constant as used herein means essentially constant.

The volume of the pressure cuff is constant according to the invention if over a respiratory cycle the volume increases or decreases by not more than 10%, preferably not more than 5%, particularly preferably not more than 2%.

The volume can also be acted on over this time of the measurement by a function with regard to a change in the volume to be chosen—this can then be recalculated again during the evaluation. Thus it is possible for example to constantly reduce the volume over the measurement and recalculate again the thus introduced changes into the measured amplitude. If the changes remain within certain tolerances and the thus introduced errors are small enough, these can also remain unconsidered in the evaluation.

In a further preferred embodiment example of the present invention, a method is provided in which the volume of the pressure cuff (20) is set in the pulsatile range of the patient such that the applied volume is chosen between the volume for ascertaining the systolic blood pressure of the patient and the volume for ascertaining the diastolic blood pressure of the patient, is preferably the average of these two values.

Preferably, for this a volume can be added to the initially sufficiently drained pressure cuff, until the first pulsatile signals can be discerned—this then prevailing volume roughly corresponds to the diastolic pressure. If further volume is now added, there is a second time at which pulsatile signals can no longer be measured—this corresponds to the systolic pressure. These values can also be ascertained in the other direction, i.e. coming from an excessive pressure it can be established when a first pulsatile signal is received (systolic pressure) and from when a signal is no longer received as volume is reduced further (diastolic pressure). If a value between these two volumes applied at these times is now used, one finds oneself in the pulsatile range. The greater the amplitudes, the more the measurement is carried out in the centre of this range, thus preferably in the average between the two volumes. The maximum amplitude of the pulsatile signals and thus the signals to be best evaluated can be expected in this range.

It is preferably also conceivable to carry out the measurement in a range below the diastolic pressure. In this case, fluctuations in calibre and a hydraulic coupling of the vessel to the outer media are still present, but non-linear effects that could result from intermittent collapse of the vessel are avoided. In relation to the diastolic pressure, there is a particularly preferred range at 0.5 to one times the diastolic pressure, particularly preferably 0.6 to 0.95 times the diastolic pressure, particularly preferably 0.7 to 0.95 times the diastolic pressure, particularly preferably 0.75 to 0.9 times the diastolic pressure, particularly preferably 0.8 to 0.9 times the diastolic pressure. Particularly preferably, the range is above the venous pressure, particularly preferably above 10 mmHg, particularly preferably above 20 mmHG, particularly preferably above 30 mmHg. Quite particularly preferably, the measurement is carried out in a range from 10 mmHg to 50 mmHg, preferably in a range from 20 mmHg to 45 mmHg, particularly preferably in a range from 25 mmHg to 40 mmHg.

Ideally, for the above-described “dynamic” measurement during inflation and deflation, a non-disruptive pressure source is avoided, i.e. the cuff is not directly filled with fluid or gas by a pump, but the cuff is supplied either from an external pressure source or a pressure tank of sufficient capacity located in the control device which is acted upon by pressure again in phases of non-measurement either from outside or by an internal pump.

During a measurement, this average can preferably be maintained by post-regulation, particularly preferably also by closing the supply lines for the fluid or the air to the pressure cuff, in order that during the measurement a constant volume which essentially does not change during the measurement is applied in the cuff

In an embodiment, a device for the non-invasive determination of in particular dynamic cardiopulmonary interaction parameters (CIPs) in a (ventilated) patient includes a pressure cuff (20) equipped for measuring the cuff pressure in the pulsatile range over at least one breathing cycle of the patient and a control device (10) for detecting the measured values of the pressure cuff (20) and for evaluating the measured pulsatile signals for ascertaining the cardiopulmonary interaction parameters (CIPs).

The preferably pneumatically or hydraulically operated pressure cuff is used as described above. The cuff pressure is preferably measured in the pulsatile range and delivers the corresponding pressure measured values via a pressure sensor in the fluid (the liquid) or in the air of the cuff.

A control device stores and evaluates the ascertained pressure measured values over time. Preferably, a processing unit such as a microprocessor or a computer is used for this. Preferably, a memory, at least a volatile memory, is also provided.

The measured values of the pressure cuff are detected via a pressure sensor in the filling medium of the cuff. These are ascertained over time.

The evaluation of the measured pulsatile signals preferably comprises the allocation of the signals over time to a heartbeat cycle as well as to a breathing cycle.

Within a breathing cycle, the cardiopulmonary interaction parameters (CIPs) can then be ascertained by comparing the absolute and relative fluctuation.

In a further preferred embodiment example of the present invention, a device is provided in which an output device (15) is provided for outputting the ascertained cardiopulmonary interaction parameter (CIP).

The output device can comprise a display or a device for transmitting the measured values or the evaluation of the ascertained cardiopulmonary interaction parameter to another unit. It is thus possible to display the value on a monitor and/or relay it to another device via an interface.

In a further preferred embodiment example of the present invention, a device is provided in which a volume-regulating device (25) is provided for regulating the volume in the pressure cuff (20).

The volume-regulating device is a device via which filling medium can be fed to or removed from the pressure cuff.

In this way, the volume in the pressure cuff can be regulated, i.e. volume can be added or removed for the measurement of the marginal values or the average pressure can be set for optimum measurement.

Measures for improving the signal-to-noise quality can preferably be taken:

An averaging over several respiratory cycles can take place.

In principle, the measurement period over which a pressure cuff can be placed under pressure on an extremity is limited because of the adverse effect on the blood circulation. However, measurement periods over several minutes are possible without having to fear damage. With longer measurement periods, it is to be borne in mind that by expressing interstitial fluid a degree of loss of pressure in the cuff is to be recorded which is preferably compensated for either by a corresponding regulation or also by appropriate numerical methods.

Measures for improving the pressure measurement quality can also preferably be taken:

During conventional oscillometric blood pressure measurement, the requirements to be met in terms of temporal resolution and correctness of the measurement are comparatively small. Pneumatic systems with pressure measurement sensors remote from the cuff in the device are therefore customarily used. An improvement in the quality of the measurement signal is to be achieved for example by using a hydraulic medium. Preferably, pressure sensors which are integrated into the cuff are also used.

The use of a material having the smallest possible extensibility for the pressure cuff and the attached tube system is likewise preferred, as an attenuation of the pulsatile amplitudes in the system itself is thereby avoided.

Furthermore, the outermost casing of the cuff is preferably rigid in design. The fluctuations in calibre of the arterial vessels are thus even more comprehensively converted into pressure fluctuations of the cuff Particularly preferably, the outer casing is rigid and the filling medium of the cuff is incompressible. This then leads to a complete coupling of the arterial vessels via the bodily tissue which is almost incompressible in relation to the necessary measurement times (that is as long as the venous vessels are empty and there is no air between the arteries and the cuff both of these are the case). The pressure can still escape laterally into the tissue. A wider cuff is preferably chosen, in particular a cuff with a width of half the circumference spanned by the cuff, preferably the whole circumference spanned by the cuff, particularly preferably of more than the circumference spanned by the cuff, in particular 1.3 to 1.5 times the circumference spanned by the cuff. Thus, the wider the cuff, the less the pressure can escape laterally.

Particularly preferably, the outer casing is completely rigid and does not just have a non-extendable outer membrane. Changes in pressure can thus also not be only partially converted into changes of the outer shape. A complete outer rigidity could be achieved with the same principle as in the case of the stiffening of vacuum mattresses, thus with an outer chamber which is filled with e.g. polystyrene microbeads and which is evacuated after being fitted. However, other possibilities for effecting a fast outer rigidity are also conceivable, such as for example the use of ultra-fast 2-component systems for the outer layer of the cuff which can effect a rigidity after activation.

In the case of a “long-sleeved” multi-chamber cuff, the pulse wave propagation velocity can preferably also be measured. In addition it is to be expected that, when the reading in the (multi-chamber) cuff is below the systolic pressure, the pulsation begins principally in the proximal cuffs, as the arterial vessels are not opened over the whole length when the reading is only just below the pressure. This can be used to identify the systolic blood pressure value. In the case of a multi-chamber long-sleeved cuff, the centrally positioned parts can be controlled like a conventional oscillometric cuff in order to identify the systolic and diastolic (average) blood pressure for the calibration, in order thus to then calibrate the signal measured over the whole length.

The described embodiments of the cuff can be carried out both in a reusable but also in a “disposable blood pressure cuff” usable for only one patient. A high precision of the measurement method with the “disposable blood pressure cuff” can be achieved by integrating an electronic pressure pick-up directly into the cuff at the place where the greatest pressure fluctuations are to be expected. This can also be achieved by a two-chamber disposable cuff where the gas volume is varied correspondingly in the outer chamber, whereas in the inner fluid-filled chamber with lower compliance which couples directly to the tissue to be compressed the pressure is measured directly with the integrated preferably electronic pressure pick-up.

Preferably, a conventional NIBP (non-invasive blood pressure) device (as is present in most patient monitors) is used as pressure cuff and the modification of the rate of the release of the pressure from the cuff is preferably carried out by an additional device comprising an additional valve. For example, it is possible to control the additional valve remotely and to reduce the otherwise too-rapid release rate of the conventional NIBP device only until a PPV value is obtained.

The fall in pressure in the cuff can thus be prevented by a specific controlled valve in co-operation with a conventional NIBP device after the cuff has been inflated. Preferably, a pressure sensor can also be integrated in the additional valve for the measurement according to the invention.

Measures during the evaluation of the pulsatile oscillations are also preferred.

The minima and maxima are preferably ascertained after artefact recognition. The surfaces under the oscillatory fluctuations can also be evaluated and here again their respiratory variation range.

In a further characteristic feature, the measured signals can be adapted to model curves, e.g. with linear or non-linear fitting methods. The sought variables can then be derived from the parameters of the model curves.

Furthermore, the standard deviation of the oscillatory fluctuations during one heartbeat or during several heartbeats or in a sliding time window (e.g. two seconds) can be evaluated. Errors due both to the slow fall in pressure and to short-term disruptions are thereby largely eliminated.

It is particularly preferably provided to use a combination of the previously named methods.

### EXAMPLE

The invention will be described in an embodiment with the help of the following example.

The cuff with pressure sensor is fitted on the upper arm of the patient.

The cuff is filled by means of a pump until the pressure fluctuations are at their maximum.

The pressure in the cuff is recorded every 10 ms during a measurement period of 30 seconds, i.e. a total of 3000 pressure values. Alternatively a longer measurement period of e.g. 90 seconds can also be chosen if a better signal is desired because of the restricted signal-to-noise ratio at low respiratory tidal volumes. Longer averaging periods can also be realized with this, for example 1-2 minutes.

The standard deviation is formed over a sliding 2 second window. With a 10 ms sampling period, this means 200 pressure values.

S(t)=standard deviation (P[t,t+2s]).

This is repeated for each sampled value t from zero to (30−2) seconds. This results in a list with 2800 standard deviations.

The maximum value Smax and the minimum value Smin is sought in the list S(t).

The pulse pressure variation PPV is calculated using

PPV=200%*(Smax−Smin)/(Smax+Smin)

### BRIEF DESCRIPTION OF THE DRAWINGS

The invention will now be further illustrated with the help of drawings. There are shown in:

FIG. 1 a curve of the temporal course of the filling of a pressure cuff according to an embodiment example of the present invention;

FIG. 2 a curve of the pulsatile measured values over at least one breathing cycle and

FIG. 3 a schematic view of a device for the non-invasive determination of cardiopulmonary interaction parameters according to an embodiment example of the present invention.

### DETAILED DESCRIPTION

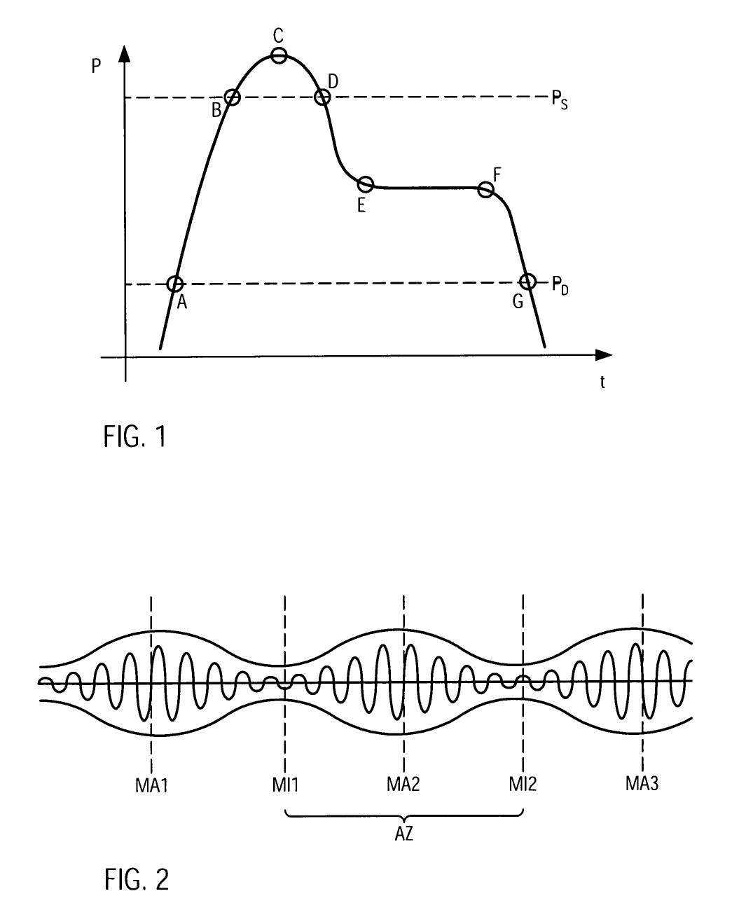

FIG. 1 shows a curve of the temporal course of the filling of a pressure cuff according to an embodiment example of the present invention. In this diagram the pressure P is plotted against time t. The diastolic pressure level PD and the systolic pressure level PS are shown by two dashed lines. The course of the pressure measured in the pressure cuff over the measurement is represented by a continuous line—points A to G are shown here for easier orientation.

The drained pressure cuff is fitted on the upper arm of a patient and filled with fluid. The pressure measured in the pressure cuff is thus increased. At point A, the pressure reaches the level of the diastolic pressure PD exerted on the upper arm and pulsatile signals are now to be recorded via the pressure sensor in the pressure cuff. The volume in the pressure cuff is further increased and the pulsatile signals first become stronger and then weaker again. At point B, the pressure reaches the level of the systolic pressure PS exerted on the upper arm and no pulsatile signals are now to be recorded via the pressure sensor in the pressure cuff. In order to make sure that the systolic pressure PS has been reached, the volume in the pressure cuff is increased a little more until point C and then the volume in the cuff is released. At point D, pulsatile signals are again recorded for the first time and the level of the systolic pressure is thus confirmed. Thus, the systolic and the diastolic levels are ascertained. Should there still be doubts as to the diastolic level, it is possible to further release the volume in the cuff until the pulsatile signals can no longer be recorded—then the diastolic level would be definitively confirmed. Starting from point D, the volume in the cuff is now further released to a level between the systolic and diastolic levels—this is reached at point E. In this range, the amplitude of the pulsatile signals is at its highest and thus the pulsatile signals to be measured are best to be picked up. At point E, the feed of fluid into the cuff is now stopped or the inlets blocked, with the result that the volume in the pressure cuff remains constant. The measurement of the pulsatile signals now takes place over at least one, preferably at least three, breathing cycles until point F. If, during this measurement, the pressure drops because of the expulsion of bodily fluid from the tissue located under the pressure cuff on the upper arm of the patient, the volume in the cuff is preferably replenished to the extent that the level between points E and F is reached again. The thus-ascertained values are evaluated after eliminating artefacts per heartbeat and per breathing cycle and the desired dynamic cardiopulmonary interaction parameters, in particular the pulse pressure variation PPV, are ascertained. The volume in the cuff is now further released and in the process passes through point G which indicates that the diastolic level PD has been reached. The pressure cuff now no longer exerts a noteworthy pressure on the upper arm and the bodily fluids expelled by the measurement can again be repositioned into the tissue. If desired, a second measurement can now be carried out following the same pattern. In a variant, it is also possible during the measurement between points E and F to discharge the fluid from the cuff in a targeted manner, in order to allow the tissue to regenerate again and then to increase the volume of the fluid back to the level E-F, in order to continue with the measurement over a further breathing cycle. In this way, the oscillations can be improved and the measurements thus carried out more reliably if the signals were to become too weak due to the exerted pressure on the upper arm during a measurement cycle.

FIG. 2 shows a curve of the pulsatile measured values over at least one breathing cycle. The measured pulsatile pressure course is represented schematically together with an envelope. The points labelled MI indicate a minimum amplitude within a breathing cycle and MA indicates the maximum values for the amplitude within the breathing cycle. AZ denotes an interval of one breathing cycle. The measurement is carried out at constant volume in the pressure cuff and shows the respiratory fluctuation of the pulsatile signals within the breathing cycle. In a first breathing cycle, the minimum is indicated by MI1 and the maximum by MA1, in a second breathing cycle by MI2 and by MA2, etc. Within the thus-identified breathing cycle, this respiratory fluctuation can now be evaluated and the desired dynamic cardiopulmonary interaction parameters, in particular the pulse pressure variation PPV, ascertained.

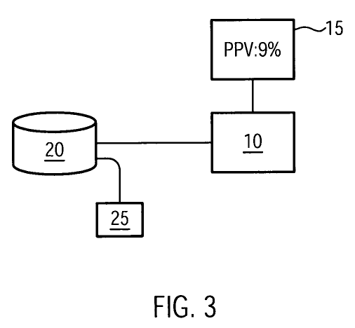

FIG. 3 shows a schematic view of a device for the non-invasive determination of cardiopulmonary interaction parameters according to an embodiment example of the present invention. A pressure cuff 20 is equipped with a volume-regulating device 25. The pressure cuff 20 preferably has an outer surface with low elasticity in order to keep the compliance low during the measurement. This can for example be realized via a non-elastic band in the outer area of the pressure cuff 20. A fluid can be fed to or removed from the pressure cuff 20 via this volume-regulating device 25. The pressure cuff 20 has a pressure sensor 21 which can detect the pressure prevailing in the pressure cuff. The pressure cuff 20 or the pressure sensor 21 within the pressure cuff 20 is connected to a control device 10 via an electrical line. In this way, the signals ascertained by the pressure sensor 21 can be sent to the control device 10. An output device 15 is attached to the control device 10.

If a measurement is now to be carried out following the course according to FIG. 1, the pressure cuff 20 is filled with fluid via the volume-regulating device 25. After passing through point A from FIG. 1 pulsatile signals which are sent to the control device 10 are detected via the pressure sensor 21. In this way, it is established by the control device that the diastolic level was reached. The volume is further increased and the measured pulsatile signals increase in intensity before they decrease again and then completely vanish when the systolic level is reached. The volume-regulating device 25 now reduces the inflow of the fluid and as a result releases the volume of the fluid in the cuff 20 to the average between the volumes which had been recorded at the diastolic and systolic levels. Point E in FIG. 1 is now reached. The volume is now kept constant by the volume-regulating device 25, i.e. the supply of fluid into the cuff 20 is blocked. The measurement is now continued over several breathing cycles at this volume level. Every second, 50 to 200 measured values, preferably 100 measured values, of the pressure sensor 21 are recorded and transmitted to the control device 10. There, the measured values are evaluated for heartbeat and breathing cycle and the minima and maxima of the amplitudes within a breathing cycle are determined. The respiratory variation of the desired dynamic cardiopulmonary interaction parameters, in particular the pulse pressure variation PPV, is ascertained from this. The thus-ascertained value is then displayed on the output device 15, in the present case a PPV of 9%.

### LIST OF REFERENCE NUMBERS

10 control device
15 output device
20 pressure cuff
21 pressure sensor
25 volume-regulating device

1-10. (canceled)

11. A method for a non-invasive determination of cardiopulmonary interaction parameters in a patient comprising:
fitting a pressure cuff on the patient;
setting a volume of the pressure cuff in a pulsatile range of the patient;
measuring pulsatile signals over time; and
evaluating the measured pulsatile signals so as to ascertain the cardiopulmonary interaction parameters.

12. The method as recited in claim 11, wherein the cardiopulmonary interaction parameters include at least one of a stroke volume variation, a pulse pressure variation and a pre-ejection phase variation.

13. The method as recited in claim 11, wherein the measuring is performed over at least one breathing cycle of the patient,

14. The method as recited in claim 11, wherein the measuring is performed over at least three breathing cycles of the patient.

15. The method as recited in claim 11, further comprising ascertaining a respirator variation range of the cardiopulmonary interaction parameters.

16. The method as recited in claim 11, wherein the measuring is performed while keeping the volume of the pressure cuff constant.

17. The method as recited in claim 11, wherein the setting of the volume includes setting the volume between a volume for ascertaining a systolic blood pressure of the patient and a volume for ascertaining the diastolic blood pressure of the patient.

18. The method as recited in claim 17, further comprising deriving a value for performing a pulse contour method from the pulsatile signals.

19. A device for a non-invasive determination of cardiopulmonary interaction parameters in a patient comprising:
a pressure cuff configured to measure a cuff pressure in a pulsative range of the patient over at least one breathing cycle of the patient; and
a control device configured to detect a measured cuff pressure value and to evaluate a measured pulsatile signal so as to ascertain the cardiopulmonary interaction parameters.

20. The device as recited in claim 19, further comprising an output device configured to output the cardiopulmonary interaction parameters.

21. The device as recited in claim 20, further comprising a volume-regulating device configured to regulate a volume in the pressure cuff.
